(19) United States
(12) Patent Application Publication
(10) Pub. No.: US 2021/0350883 A1
(43) Pub. Date: Nov. 11, 2021

(54) USER AND TEST SAMPLE IDENTIFICATION/AUTHENTICATION SYSTEM FOR CONTROL OF INFECTIOUS DISEASE PROPAGATION

(71) Applicants: Shu Li, Irvine, CA (US); Xiping Wu, Irvine, CA (US)

(72) Inventors: Shu Li, Irvine, CA (US); Xiping Wu, Irvine, CA (US)

(73) Assignee: J&J Investments Worldwide, Ltd., Irvine, CA (US)

(21) Appl. No.: 16/868,031

(22) Filed: May 6, 2020

(51) Int. Cl. G16H 10/40; G06Q 30/06; G16H 50/20; G06Q 50/26; G16H 15/00; H04L 29/06; G06K 7/14; C12Q 1/6876; G06K 9/00

(52) U.S. Cl. CPC G16H 10/40; G06Q 30/0633; G16H 50/20; G06Q 50/265; G16H 50/80; H04L 63/0884; G06K 7/1417; C12Q 1/6876; G06K 9/00134; G16H 15/00

(57) ABSTRACT

A user and test sample authentication system and method for identifying COVID-19 infected users is disclosed. An authentication platform receives a first user profile data from a first user device to register to an authentication service and generate a first scannable image. The platform receives a second user profile data from the second user device for registration and generates a second scannable image. A purchase order of a medical kit is placed to perform one or more diagnosis procedures on the first user to identify if the first user is infected by COVID-19. The diagnosis data is encoded to the first scannable image to form a unique corona ID. On scanning the unique ID, the diagnosis data and the user profile data are displayed, whereby the presence of coronavirus infection of the first user is determined before contacting one or more individuals to prevent spread of the disease or infection.

### BACKGROUND OF THE INVENTION

### A. Technical Field

The present invention generally relates to preventing infectious disease (such as Cov-19) propagation, and more specifically relates to a user and test sample identification and authentication system which gives users viral-test negative identification so as to participate in work and social places safely.

### B. Description of Related Art

Coronvirus disease (COVID-19) is an infectious disease caused by a newly discovered coronavirus. Most people infected with the COVID-19 virus experiences mild to moderate respiratory illness. Older people, and those with underlying medical problems like cardiovascular disease, diabetes, chronic respiratory disease, and cancer are more likely to develop serious illness and results in death. The primary way that COVID-19 appears to spread is by close person-to-person contact.

Most cases of COVID-19 have involved people who cared for or lived with someone with COVID-19, or had direct contact with infectious material (for example, respiratory secretions) from a person who has COVID-19. Potential ways in which COVID-19 can be spread include touching the skin of other people or objects that are contaminated with infectious droplets and then touching your eye(s), nose, or mouth. This can happen when someone who is sick with COVID-19 coughs or sneezes droplets onto themselves, other people, or nearby surfaces. Presently, no solution is available to prevent the rapid spread of infection other than self-isolation, which has significantly disrupted the society and the economy due to the requirement of self-isolation/social distancing. Another key issue is that people infected don't show symptoms until after the incubation period and some may never show symptom.

Few existing patent references related to the above-discussed problem are discussed as follows.

US20200023360 of Cunningham Brian T entitled “system for Rapid, Portable, and Multiplexed Detection and Identification of Pathogen Specific Nucleic Acid Sequences” discloses sample carrier including a sample preparation module and an amplification module. A sample mixes with a lysis medium and a nucleic acid amplification medium in the sample preparation module and then flows into a plurality of microfluidic chambers in the amplification module. The microfluidic chambers have disposed therein primers configured to initiate amplification of one or more target nucleic acid sequences corresponding to one or more pathogens. The sample carrier is inserted into an apparatus that includes a plurality of Sight sources and a camera. The light sources illuminate the microfluidic chambers with excitation light, a fluorophore emits fluorescence light indicative of nucleic acid amplification in response to the excitation-light, and the camera captures images of the microfluidic chambers. A target nucleic acid sequence in the sample is indicated by the images showing an increasing fluorescence in a microfluidic chamber that has the primers for that sequence.

U.S. Pat. No. 9,811,818 of Xing Zhou Tian entitled “wearable personal digital device for facilitating mobile device payments and personal use” discloses a wearable personal digital device for point of healthcare saliva testing. The wearable personal digital device may comprise a processor, a display, biometric sensors, activity tracking sensors, a memory unit, a communication circuit, a housing, an input unit, a projector, a timepiece unit, a haptic touch control actuator, a band, a mounting clip, a saliva sample insert apparatus, a pinhole, a light emitting diode board, a battery, and a set of sensors. The processor may be operable to receive data from an external device, provide a notification to a user based on the data, receive a user input, and perform a command selected based on the user input. The housing may be adapted to enclose the components of the wearable personal digital device. The band may be adapted to attach to the housing and secure the wearable personal digital device on a user body. However, the above discussed references do not authenticate the user nor the sample data is indeed from that user.

Further the above discussed references lack to provide a solution to prevent the spread of COVID-19. Therefore, there is a need for a user and test sample authentication system and method for identifying coronavirus infection/disease and preventing the spread of the virus.

### SUMMARY OF THE INVENTION

The present invention discloses a user and test sample authentication system and method for identifying coronavirus infection or disease, or COVID-19 infected users.

The system comprises a first user device associated with a first user, a second user device associated with a second user and an authentication platform in communication with the first user device and the second user device via a network. The authentication platform comprises an authentication server and at least one database. An authentication platform application programming interface (API) of the platform facilitates communication of the authentication platform with the first user device, and the second user device. The platform is configured to provide a unique ID incorporated with the diagnosis data to identify if the user of the unique ID is infected with coronavirus. In one embodiment, the first user could be any individual who requires the unique ID. In one embodiment, the second user is a health care professional.

The authentication server is configured to receive a first user profile data from the first user device to register to the authentication service of the authentication platform. The authentication platform is further configured to generate a first scannable image encoded with at least a part of first user profile data. The first user profile data, includes, but not limited to, an email address, data identifying the first user device, data identifying the first user, and image data of the first user, a scanned identification (driver's license, passport) of the first user, a photo taken of the first user. On receiving the first user profile data, a one-time password is sent to the user for identity verification and, thereafter, the first scannable image is generated.

The authentication server is further configured to receive a second user profile data from the second user device to register to the authentication service of the authentication platform. The authentication server is further configured to generate a second scannable image encoded with at least a part of second user profile data. The second user profile data, includes, but not limited to, an email address, data identifying the second user device, data identifying the second user, image data of the first user and data identifying a profession of the second user. On receiving the second user profile data, a one-time password is sent to the user for identity verification and, thereafter, the second scannable image is generated.

The authentication server further facilitates to place a purchase order of a medical kit to perform one or more diagnosis procedures on the first user. The diagnosis procedure enables to identify if the first user is infected by the coronavirus. The second user, classified by the authentication server as a medical professional, shall witness or perform, either virtually by video conferencing or physically in person, the diagnostic test to the first user.

In one embodiment, the first user or the second user could draw his/her sample (e.g., saliva) into a collection tube, wait for the sample and testing solution to react. Thereafter, the resulting assay shall be microscoped, and the picture of the reacted sample is digitally analyzed by computer AI algorithm. This could be either be performed by the user, or by the platform (using smartphone). In another embodiment, the server is incorporated with set of instructions/modules to scan the assay and analyze the picture through its AI algorithm to register the test results/diagnosis data into the platform.

In another embodiment, the diagnosis data of the first user for coronavirus infection is uploaded at the authentication platform. The diagnosis data is uploaded by entering the first scannable image and the second scannable image simultaneously for authentication. Further, the diagnosis data is encoded to the first scannable image of the first user. The first scannable image encoded with the diagnosis data is referred as unique ID/unique corona ID. On scanning the unique ID, the diagnosis data and the user profile data are displayed, whereby the presence of coronavirus infection of the first user is determined at an event before contacting one or more individuals to prevent spread of the infection/disease. The unique ID could be only used for a predefined time period and has to be renewed by performing the test for detecting the coronavirus infection. The unique ID maintenance scheme ensures the user continues to be tested within any incubation period, for example, for COVID-19, every seven (7) days, in order to maintain a test negative status.

In yet another embodiment, the system could also be operated as a single user system. The user, either of a first user or a second user, could order the test kit and perform the test. Then, the platform along with a smartphone with or without an adaptive lens/device is utilized to scan the assay of the test, then the app shall analyze the picture through its AI algorithm to register the test results into the platform. Additionally, the application/authentication service enables to scan, possibly use a different adaptive lens/device such as a portable cell phone mountable spectrometer, the assay to identify the assay is indeed from to the user's body. In some embodiments, the test is not only intended to test people, but also environment, i.e., the samples can be from environment (air, surfaces, etc.).

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF DRAWINGS

The embodiments herein will be better understood from the following detailed description with reference to the drawings, in which:

FIG. 1 exemplarily illustrates an environment incorporated with a user and sample authentication system for identifying coronavirus infection, according to an embodiment of the present invention.

FIG. 2 exemplarily illustrates a components and connection between the components of the authentication server, according to an embodiment of the present invention.

FIG. 3 exemplarily illustrates a user authentication method for identifying coronavirus infection, according to an embodiment of the present invention.

FIG. 4 exemplarily illustrates a screenshot of a user interface displaying a unique scannable image, according to an embodiment of the present invention.

FIG. 5 exemplarily illustrates a screenshot of a user interface displaying the status to determine corona virus infection, according to an embodiment of the present invention.

FIG. 6 exemplarily illustrates a screenshot of a user interface to perform diagnosis procedure to identify if the user is infected by the coronavirus, according to an embodiment of the present invention.

FIG. 7 exemplarily illustrates a screenshot of a user interface displaying instructions to perform diagnosis procedure, according to an embodiment of the present invention.

### DETAILED DESCRIPTION OF EMBODIMENTS

A description of embodiments of the present invention will now be given with reference to the Figures. It is expected that the present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

As will be appreciated by one skilled in the art, aspects of the embodiments may be embodied as a device, system, method or program product. Many of the functional units described in this specification have been labeled as modules, in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in code and/or software for execution by various types of processors. An identified module of code may, for instance, comprise one or more physical or logical blocks of executable code which may, for instance, be organized as an object, procedure, or function. Nevertheless, the executables of an identified module need not be physically located together, but may comprise disparate instructions stored in different locations which, when joined logically together, comprise the module and achieve the stated purpose for the module.

Furthermore, the described features, structures, or characteristics of the embodiments may be combined in any suitable manner. In the following description, numerous specific details are provided, such as examples of programming, software modules, user selections, network transactions, database queries, database structures, hardware modules, hardware circuits, hardware chips, etc., to provide a thorough understanding of embodiments. One skilled in the relevant art will recognize, however, that embodiments may be practiced without one or more of the specific details, or with other methods, components, materials, and so forth. In other instances, well-known structures, materials, or operations are not shown or described in detail to avoid obscuring aspects of an embodiment.

Aspects of the embodiments are described below with reference to schematic flowchart diagrams and/or schematic block diagrams of methods, apparatuses, systems, and program products according to embodiments. It will be understood that each block of the schematic flowchart diagrams and/or schematic block diagrams, and combinations of blocks in the schematic flowchart diagrams and/or schematic block diagrams, can be implemented by code. These codes may be provided to a processor or other programmable data processing apparatus to produce a machine, such that the instructions, which execute via the processor or other programmable data processing apparatus, create means for implementing the functions/acts specified in the schematic flowchart diagrams and/or schematic block diagrams block or blocks.

The schematic flowchart diagrams and/or schematic block diagrams in the Figures illustrate the architecture, functionality, and operation of possible implementations of apparatuses, systems, methods and program products according to various embodiments. In this regard, each block in the schematic flowchart diagrams and/or schematic block diagrams may represent a module, segment, or portion of code, which comprises one or more executable instructions of the code for implementing the specified logical function(s).

It should also be noted that, in some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. Other steps and methods may be conceived that are equivalent in function, logic, or effect to one or more blocks, or portions thereof, of the illustrated Figures.

Although various arrow types and line types may be employed in the flowchart and/or block diagrams, they are understood not to limit the scope of the corresponding embodiments. Indeed, some arrows or other connectors may be used to indicate only the logical flow of the depicted embodiment. For instance, an arrow may indicate a waiting or monitoring period of unspecified duration between enumerated steps of the depicted embodiment. It will also be noted that each block of the block diagrams and/or flowchart diagrams, and combinations of blocks in the block diagrams and/or flowchart diagrams, can be implemented by special purpose hardware-based systems that perform the specified functions or acts, or combinations of special purpose hardware and code.

The description of elements in each figure may refer to elements of proceeding figures. Like numbers refer to like elements in all figures, including alternate embodiments of like elements.

The present invention discloses a user and test sample authentication system and method for identifying coronavirus infection or disease, or Coronavirus Disease of 2019 (COVID-19) infected users. The system is configured to determine if a user has coronavirus infection to prevent the spread of the infection. The system is configured to provide a unique ID incorporated with the diagnosis data to identify if the user of the unique ID is infected with coronavirus. The system could be used at a social event to determine if any of the attendee of the event has the coronavirus infection before joining the event, thereby prevents spread of the infection.

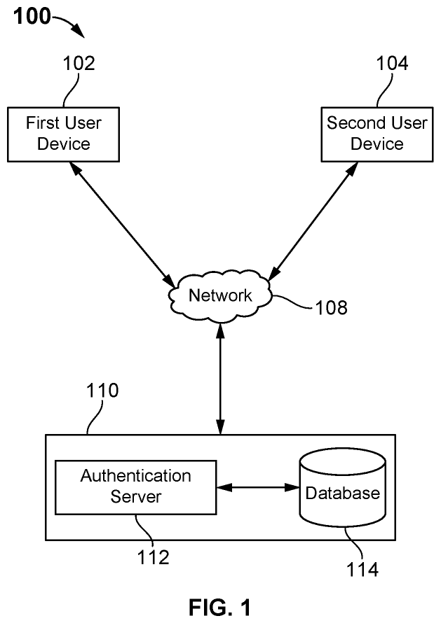

FIG. 1 exemplarily illustrates an environment 100 incorporated with the authentication system for identifying coronavirus infection, according to an embodiment of the present invention. The system comprises a first user device 102 associated with a first user, a second user device 104 associated with a second user, and an authentication platform 110 in communication with the first user device 102, and the second user device 104, via a network 108. The authentication platform 110 comprises an authentication server 112 and at least one database. An authentication platform application programming interface (API) of the platform 110 facilitates communication of the authentication platform 110 with the first user device 102, and the second user device 104. The platform 110 is configured to provide a unique ID incorporated with the diagnosis data to identify if the user of the unique ID is infected with coronavirus.

In one embodiment, the first user could be any individual who requires the unique ID. In one embodiment, the second user is a health care professional.

The user devices (102, 104) are a computing device configured to access the services provided by the server 112. User devices (102, 104) further have the capability to provide the user an interface to interact with the services provided by the server 112. User devices (102, 104) may be, for example, a desktop computer, a laptop computer, a mobile phone, a personal digital assistant, and the like. User devices (102, 104) may execute one or more client applications such as, without limitation, a web browser to access and view content over a computer network, an email client to send and retrieve emails, an instant messaging client for communicating with other users, and a File Transfer Protocol (FTP) client for file transfer. User devices (102, 104), in various embodiments, may include a Wireless Application Protocol (WAP) browser or other wireless or mobile device protocol suites. User devices (102, 104) are incorporated with an image capture module.

Network 108 generally represents one or more interconnected networks, over which the user devices and server 112 can communicate with each other. Network 108 may include packet-based wide area networks (such as the Internet), local area networks (LAN), private networks, wireless networks, satellite networks, cellular networks, paging networks, and the like. A person skilled in the art will recognize that network 108 may also be a combination of more than one type of network. For example, network 108 may be a combination of a LAN and the Internet. In addition, network 108 may be implemented as a wired network, or a wireless network or a combination thereof.

In an embodiment, the at least one database 114 may be accessible by the authentication server 112. In another embodiment, the at least one database 114 may be integrated into the authentication server 112 or separate from it. In an embodiment, the at least one database 114 resides in a connected server or in a cloud computing service. In an embodiment, regardless of location, the at least one database 114 comprise a memory to store and organize certain data for use by the authentication server 112. In one embodiment, the at least one database 114 stores profile data and diagnosis data of the first user, and the second user. The at least one database 114 further stores information to diagnose the coronavirus infection. In one embodiment, the profile data comprises email address, data identifying the user, image of the user, data identifying the profession of the user.

In one embodiment, the authentication server 112 is at least one of a general or special purpose computer. In an embodiment, it operates as a single computer, which can be a hardware and/or software server, a workstation, a desktop, a laptop, a tablet, a mobile phone, a mainframe, a supercomputer, a server farm, and so forth. In an embodiment, the computer could be touchscreen and/or non-touchscreen device and could run on any type of OS, such as iOS™, Windows™, Android™, Unix™, Linux™ and/or others. In an embodiment, the computer is in communication with network 108. Such communication can be via a software application, a mobile app, a browser, an OS, and/or any combination thereof.

The authentication server 112 is configured to receive a first user profile data from the first user device 102 to register to the authentication service of the authentication platform 110. The authentication platform 110 is further configured to generate a first scannable image encoded with at least a part of first user profile data. The first user profile data, includes, but not limited to, an email address, data identifying the first user device 102, data identifying the first user, and image data of the first user. On receiving the first user profile data, a one-time password is sent to the user for identity verification and, thereafter, the first scannable image is generated.

The authentication server 112 is further configured to receive a second user profile data from the second user device 104 to register to the authentication service of the authentication platform 110. The authentication server 112 is further configured to generate a second scannable image encoded with at least a part of second user profile data. The second user profile data, includes, but not limited to, an email address, data identifying the second user device 104, data identifying the second user, image data of the first user and data identifying a profession of the second user. On receiving the second user profile data, a one-time password is sent to the user for identity verification and, thereafter, the second scannable image is generated.

The authentication server 112 further facilitates to place a purchase order of one or more medical kits to perform one or more diagnosis procedures on the first user. The diagnosis procedure enables to identify if the first user is infected by the coronavirus. The diagnosis data of the first user for coronavirus infection is uploaded at the authentication platform 110. The diagnosis data is uploaded by entering the first scannable image and the second scannable image simultaneously for authentication. Further, the diagnosis data is encoded to the first scannable image of the first user.

The first scannable image encoded with the diagnosis data is referred as unique ID/unique corona ID. On scanning the unique ID, the diagnosis data and the user profile data are displayed, whereby the presence of coronavirus infection of the first user is determined at an event before contacting one or more individuals to prevent spread of the infection. The unique ID could be only used for a predefined time period and has to be renewed by performing the test for detecting the coronavirus infection. The unique ID maintenance scheme ensures the user continues to be tested within any incubation period, for example, for COVID-19, every seven (7) days, in order to maintain a test negative status. In one embodiment, the first scannable image, the second scannable image and unique ID/unique scannable image are QR codes. In some embodiments, the test is not only intended to test people, but also environment, i.e., the samples can be from environment (air, surfaces, etc.).

The authentication server 112 is configured to verify the identity of the first user using a government issued identification document and also verifies that the diagnostic data matches with the DNA of the first user. The authentication server 112 is further configured to verify the identity of the second user utilizing a government issued identification document. The government issued identification document may be a driver license, passport or any other document.

The diagnostic procedure is either self-performed by the first user by the medical kit. The medical kit is a test kit including a collection tube and a test strip. The test strip receives a sample of the first user. An image of the tested sample is captured using the image capture module and uploaded to the authentication platform 110. The image capture module is a camera or smartphone adaptable magnification lens that could be used as a microscope and photo digitizing tool. The first user device 102 is configured to act as a fluorescent spectroscopy to read samples (saliva, blood, and other body fluids) for forensic evidence.

An image of the sample prior to testing is also uploaded to the authentication platform 110. Further, the tested sample is sealed in a package and sent to a laboratory for quality control. The central lab may test the DNA of the sample in order to further certify the authenticity of the sample against the first user. To verify if the sample indeed belongs to the same individual/first user, the procedure is carried out in presence of the healthcare provider/second user. The procedure could also be carried out by connecting the healthcare provider through webcam as witness. The authentication server 112 is configured to analyses the image of the tested sample. The authentication server 112 is incorporated with artificial intelligence (AI) algorithm to analyze the image of the tested sample and determine if the individual is affected by the coronavirus. Then the determined diagnosis data is encoded to the first scannable image. In some embodiments, the test is not only intended to test people, but also environment, i.e., the samples can be from environment (air, surfaces, etc.).

In another embodiment, the diagnostic procedure is performed by the health care provider. The healthcare provider verifies and uploads the diagnosis data of the first user to the authentication platform 110. The diagnosis data is uploaded by entering the first scannable image and the second scannable image simultaneously for authentication.

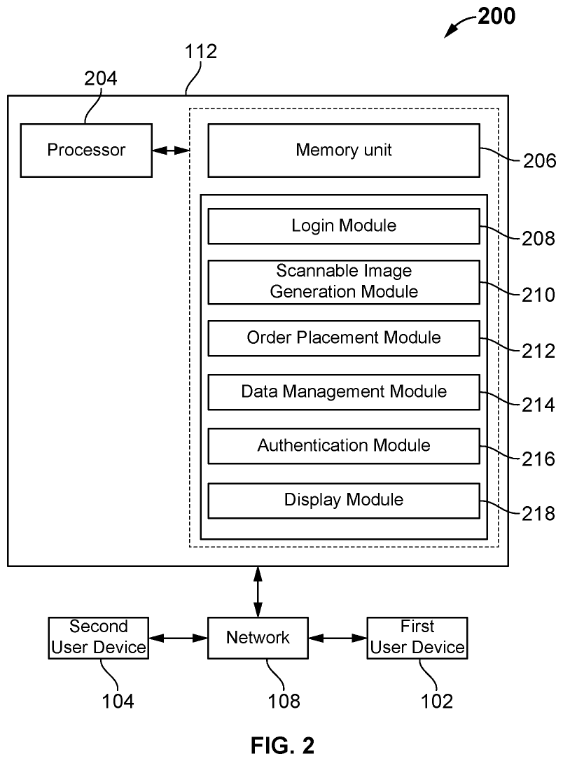

Referring to FIG. 2, a block diagram 200 of a components and connection between the components of the authentication server 112 is disclosed, according to an embodiment of the present invention. The server 112 comprises a processor 204 and a memory 206. The memory 206 stores set of program modules executable by the processor 204. The set of modules includes a login module 208, a scannable image generation module 210, an order placement module 212, a data management module 214, an authentication module 216 and the display module 218.

The login module 208 is configured to receive the first user profile data from the first user device 102 to register to the authentication service of the authentication platform 110. The scannable image generation module 210 is further configured to generate a first scannable image encoded with at least a part of first user profile data. The first user profile data, includes, but not limited to, an email address, data identifying the first user device 102, data identifying the first user, and image data of the first user. The login module 208 is further configured to receive a second user profile data from the second user device 104 to register to the authentication service of the authentication platform 110. The scannable image generation module 210 is further configured to generate a second scannable image encoded with at least a part of second user profile data. The second user profile data, includes, but not limited to, an email address, data identifying the second user device 104, data identifying the second user, image data of the first user and data identifying a profession of the second user.

The order placement module 212 facilitates to place a purchase order of one or more medical kits to perform one or more diagnosis procedures on the first user. The diagnosis procedure enables to identify if the first user is infected by the coronavirus. The diagnosis data of the first user for coronavirus infection is uploaded at the authentication platform 110, via the data management module 214. The diagnosis data is uploaded by entering the first scannable image and the second scannable image simultaneously for authentication. Further, the diagnosis data is encoded to the first scannable image of the first user, via the scannable image generation module 210. The first scannable image encoded with the diagnosis data is referred as unique scannable image, unique ID or unique corona ID. On scanning the unique ID, the display module 218 facilitates to display the diagnosis data and the user profile data, whereby the presence of coronavirus infection of the first user is determined at an event before contacting one or more individuals to prevent spread of the infection. The unique ID could be only used for a predefined time period and has to be renewed by performing the test for detecting the coronavirus infection. The unique ID maintenance scheme ensures the user continues to be tested within any incubation period, for example, for COVID-19, every seven (7) days, in order to maintain a test negative status.

The authentication module 216 is configured to verify the identity of the first user using a government issued identification document and also verifies that the diagnostic data matches with the DNA of the first user. The authentication module 216 is further configured to verify the identity of the second user utilizing a government issued identification document.

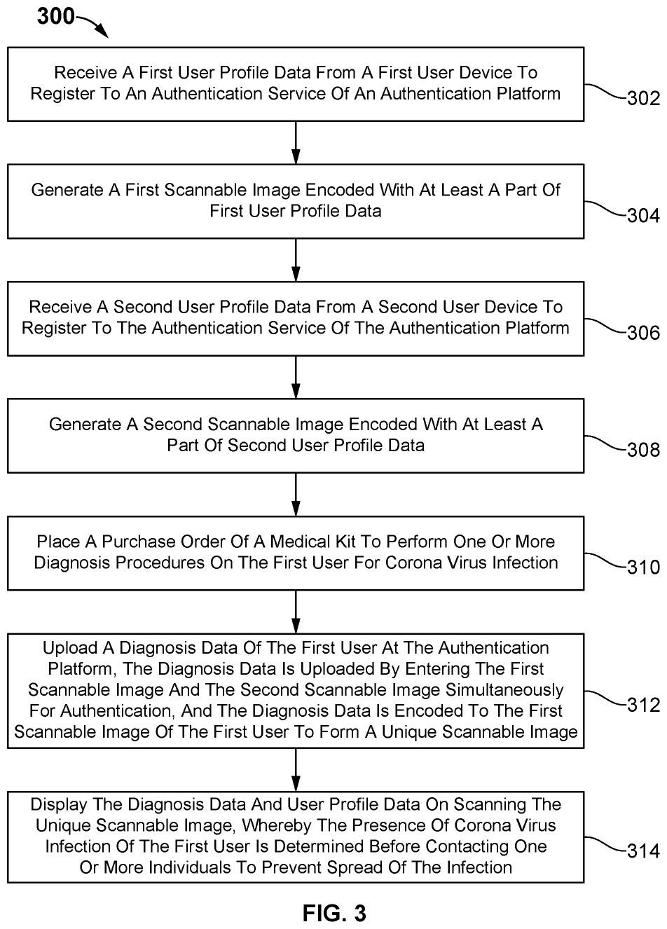

FIG. 3 exemplarily illustrates a flowchart of user authentication method 300 for identifying coronavirus infection, according to an embodiment of the present invention. The method 300 is incorporated in an authentication platform comprising at least one computing device having a processor coupled to a memory, wherein the platform is in communication with a first user device associated with a first user and a second user device associated with a second user. Each user device comprises an image capture module. The image capture module is a smartphone camera or a smartphone adaptable magnification lens.

At step 302, the authentication server receives a first user profile data from a first user device to register to an authentication service of the authentication platform. At step 304, a first scannable image encoded with at least a part of first user profile data is generated. At step 306, the authentication server receives a second user profile data from the second user device to register to the authentication service of the authentication platform. At step 308, a second scannable image encoded with at least a part of second user profile data is generated.

At step 310, a medical kit purchase order is placed. The medical kit facilitates to perform one or more diagnosis procedures on the first user for identifying coronavirus infection.

A sample of the first user is tested at home, point of care or laboratory, using the medical test kit. The test kit includes a test strip, where the sample of the individual is placed. The image capture module functions as a microscope and photo digitizing tool and the authentication server incorporated with AI algorithm to analyze the resulting picture of patient/first users' samples. The test strip kit, after reaction to the sample (e.g., saliva), could be read from the microscope, wherein the smartphone zoom lens or with a mounting lens and its camera act as the microscope involves in taking digital picture. The user device acts as fluorescent spectroscopy to read samples (saliva, blood, and other body fluids) for forensic evidence, e.g. DNA verification for authentication of the individual. In some embodiments, the test is not only intended to test people, but also environment, i.e., the samples can be from environment (air, surfaces, etc.).

In one embodiment, the system validates the sample is indeed from the same individual via the following methods. In one method, the certified health professional/second user conducts the test point of care (POC) and enters the test result to the platform by simultaneously entering the QR code of the health professional and the patients for verification.

In another method, the certified health professional observes the user (individual being tested utilizing the platform) collecting the sample and conducting the test in person or through webcam as a witness. The first user collects his/her sample using the test kit, and perform the test per instruction, and use his/her smartphone camera (at least 5× magnification) or with a mounting lens as per specification of the test to take pictures of the tested sample and/or the sample prior to testing and to transmit the resulting pictures to the platform, and/or seal the mailing package for mailing the sample to a central lab for quality control, and that the user scan his/her QR code and the health professional also scan his/her QR code.

In yet another method, a certified health professional witnesses the collection of the sample, and scan the professional and the individual QR, along with the serial code of the collection tube. The collected samples are mailed to the central lab. The central lab may test the DNA of the sample in order to further certify the authenticity of the sample against the individual.

At step 312, the diagnosis data of the first user is uploaded to the authentication platform. The diagnosis data is uploaded by entering the first scannable image and the second scannable image simultaneously for authentication. The diagnosis data is encoded to the first scannable image of the first user to form a unique scannable image.

At step 314, the diagnosis data and first user profile data are displayed on scanning the unique scannable image/unique ID, whereby the presence of coronavirus infection of the first user is determined before contacting one or more individuals to prevent spread of the coronavirus infection. The unique ID is used as a valid identification, passport or pass for people not infected by coronavirus to interact with other people who also maintained a valid unique corona ID, at workplace, events, gatherings, or any other places that requires interaction with people. The unique ID could be used at a social event to determine if any of the attendee of the event has the coronavirus infection before joining the event, thereby prevents spread of the infection. The unique ID maintenance scheme ensures the user continues to be tested within any incubation period, for example, for COVID-19, every seven (7) days, in order to maintain a test negative status.

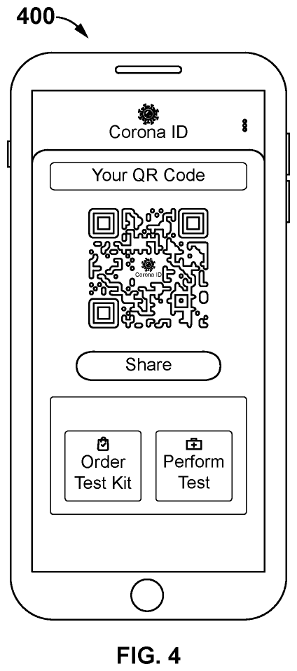

FIG. 4 exemplarily illustrates a screenshot 400 of a user interface displaying a unique scannable image, according to an embodiment of the present invention. On scanning the unique scannable image by the healthcare professional, data including, but not limited to, test history, patient/user history, order history are displayed.

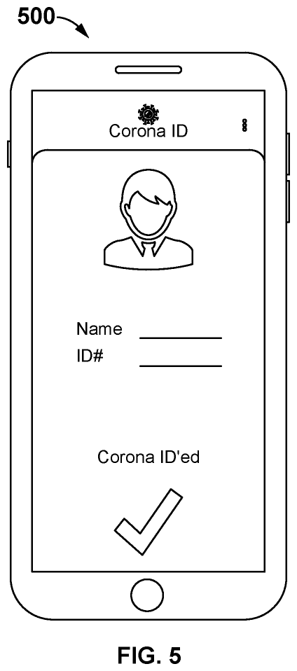

FIG. 5 exemplarily illustrates a screenshot 500 of a user interface displaying the status to determine corona virus infection, according to an embodiment of the present invention. Indica of different color are utilized to display the status of the user. If the user is tested negative and the test was done within the last 7 days, green colored indicia is displayed. If the user is tested positive and test was done within the last 7 days, red colored indicia is displayed. If the user has not renewed the test data, yellow colored indicia is displayed.

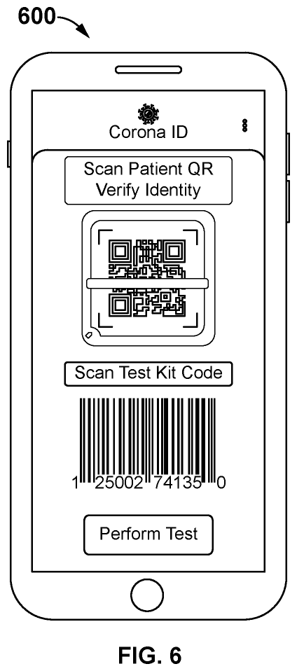

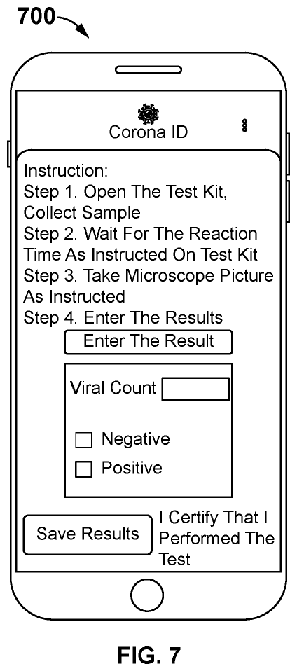

FIG. 6 exemplarily illustrates a screenshot 600 of a user interface to perform diagnosis procedure to identify if the user is infected by the coronavirus, according to an embodiment of the present invention. The user interface displays the unique scannable image/QR code and a scan test kit code to perform the test. On scanning the code, the user is redirected to the user interface shown in FIG. 7. Further, the healthcare professional cannot perform the test on himself/herself.

FIG. 7 exemplarily illustrates a screenshot 700 of a user interface displaying instructions to perform diagnosis procedure, according to an embodiment of the present invention. The instructions displayed includes, open the test kit; collect the sample; wait for the reaction time as instructed on test kit, enter and save the test results.

Although a single embodiment of the invention has been illustrated in the accompanying drawings and described in the above detailed description, it will be understood that the invention is not limited to the embodiment developed herein, but is capable of numerous rearrangements, modifications, substitutions of parts and elements without departing from the spirit and scope of the invention.

The foregoing description comprises illustrative embodiments of the present invention. Having thus described exemplary embodiments of the present invention, it should be noted by those skilled in the art that the within disclosures are exemplary only, and that various other alternatives, adaptations, and modifications may be made within the scope of the present invention. Merely listing or numbering the steps of a method in a certain order does not constitute any limitation on the order of the steps of that method. Many modifications and other embodiments of the invention will come to mind to one skilled in the art to which this invention pertains having the benefit of the teachings presented in the foregoing descriptions. Although specific terms may be employed herein, they are used only in generic and descriptive sense and not for purposes of limitation. Accordingly, the present invention is not limited to the specific embodiments illustrated herein.

What is claimed is:

1. A user and test sample authentication method for identifying coronavirus infection or disease is incorporated in an authentication platform comprising at least one computing device having a processor coupled to a memory, wherein the platform is in communication with a first user device associated with a first user and a second user device associated with a second user, the method comprises the steps of:
receiving, at the authentication platform, a first user profile data from a first user device to register to an authentication service of the authentication platform;
generating, at the authentication platform, a first scannable image encoded with at least a part of first user profile data;
receiving, at the authentication platform, a second user profile data from the second user device to register to the authentication service of the authentication platform;
generating, at the authentication platform, a second scannable image encoded with at least a part of second user profile data;
placing, at the authentication platform, a purchase order of a medical kit to perform one or more diagnosis procedures on the first user for identifying coronavirus infection;
uploading, at the authentication platform, a diagnosis data of the first user at the authentication platform, wherein the diagnosis data is uploaded by entering the first scannable image and the second scannable image simultaneously for authentication, and wherein the diagnosis data is encoded to the first scannable image of the first user to form a unique scannable image, and
displaying, at the first user device, the diagnosis data and first user profile data on scanning the unique scannable image, whereby the presence of coronavirus infection of the first user is determined before contacting one or more individuals to prevent spread of the coronavirus infection.

2. The authentication method of claim 1, wherein the diagnostic procedures are self-performed by the first user with a witness comprises the steps of:
placing a sample of the first user on a test kit;
capturing image of tested sample using an image capture module of the first user device, wherein the image capture module is a camera or smartphone adaptable magnification lens used as a microscope and photo digitizing tool;
uploading and analysing the image at the authentication server incorporated with AI algorithm to determine the diagnosis data, and
testing DNA of the sample to certify the authenticity of the sample against the first user.

3. The authentication method of claim 1, wherein the diagnostic procedures are performed by the second user, and wherein the second user verifies and uploads the diagnosis data of the first user to the authentication platform.

4. The authentication method of claim 1, further comprises a step of: verifying the identity of the first user and second user utilizing a government issued identification document.

5. The authentication method of claim 1, wherein the first scannable image, the second scannable image and the unique scannable image is a QR code.

6. The authentication method of claim 1, wherein the first user profile data comprises an email address, data identifying the first user device, data identifying the first user, and image data of the first user.

7. The authentication method of claim 1, wherein the first user is an individual.

8. The authentication method of claim 1, wherein the second user is a healthcare provider.

9. The authentication method of claim 1, wherein the second user profile data comprises an email address, data identifying the second user device, data identifying the second user, image data of the first user and data identifying a profession of the second user.

10. A user and test sample authentication system for identifying coronavirus infection, comprising:
a first user device having an image capture module associated with a first user;
a second user device having an image capture module associated with a second user, and
an authentication platform comprising at least one computing device having a processor coupled to a memory, wherein the verification platform is adapted for communication with the first user device and the second user device, and is configured to:
receive a first user profile data from the first user device to register to an authentication service of the authentication platform;
generate a first scannable image encoded with at least a part of first user profile data;
receive a second user profile data from the second user device to register to the authentication service of the authentication platform;
generate a second scannable image encoded with at least a part of second user profile data;
place a purchase order of a medical kit to perform one or more diagnosis procedures on the first user for coronavirus infection;
upload a diagnosis data of the first user at the authentication platform, wherein the diagnosis data is uploaded by entering the first scannable image and the second scannable image simultaneously for authentication, and wherein the diagnosis data is encoded to the first scannable image of the first user to form a unique scannable image, and
display the diagnosis data and user profile data on scanning the unique scannable image, whereby the presence of coronavirus infection of the first user is determined before contacting one or more individuals to prevent spread of the infection.

11. The authentication system of claim 10, is configured to facilitate one or more non-registered users to place order for the medical kit.

12. The authentication system of claim 10, wherein the diagnostic procedures are self-performed by the first user with a witness, comprises the steps of:
placing a sample of the first user on a test kit;
capturing image of tested sample using an image capture module of the first user device, wherein the image capture module is a camera or smartphone adaptable magnification lens used as a microscope and photo digitizing tool;
uploading and analysing the image at the authentication server incorporated with AI algorithm to determine the diagnosis data, and
testing DNA of the sample to certify the authenticity of the sample against the first user.

13. The authentication system of claim 10, wherein the diagnostic procedures are performed by the second user, and wherein the second user verifies and uploads the diagnosis data of the first user to the authentication platform.

14. The authentication system of claim 10, is configured to verify the identity of the first user and second user utilizing a government issued identification document.

15. The authentication system of claim 10, wherein the first scannable image, the second scannable image and the unique scannable image is a QR code.

16. The authentication system of claim 10, wherein the first user profile data comprises an email address, data identifying the first user device, data identifying the first user, and image data of the first user.

17. The authentication system of claim 10, wherein the first user is an individual.

18. The authentication system of claim 10, wherein the second user is a healthcare provider.

19. The authentication system of claim 10, wherein the second user profile data comprises an email address, data identifying the second user device, data identifying the second user, image data of the first user and data identifying a profession of the second user.
